(19) United States
(12) Patent Application Publication
(10) Pub. No.: US 2003/0088314 A1
(43) Pub. Date: May 8, 2003

(54) Alveolar bone extension apparatus

(76) Inventor: Tomoaki Koseki, Chiyoda-ku (JP)

Correspondence Address: Tomoaki Koseki, 17-2 Sotokanda 2-Chome, Chiyoda-Ku, 101-0021, JP

(21) Appl. No.: 10/286,685

(22) Filed: Nov. 1, 2002

(30) Foreign Application Priority Data
Nov. 5, 2001 (JP) JP 2001-339339

(51) Int. Cl. A61F 2/28

(52) U.S. Cl. 623/17.18

(57) ABSTRACT

An alveolar bone extension apparatus consisting of three parts, which are a plate with screw holes, an extension screw, and fixing screws, wherein the plate is fixed onto an alveolar bone basis and a movable bone fragment with fixing screws, and alveolar bone extension is enabled by rotating an extension screw through a cylinder. This apparatus has the form and structure of making the tip end of the extension screw so as to prevent the end from digging into the alveolar bone basis. Also the thread cutting pitch of the extension screw is set to match with one day's quantity of desired bone extension volume. The holes on the plate are arranged in two rows, one row for odd number holes and the other for even number holes, the two rows lined alternately so as to make the distance between the two rows as short as possible to enable the size of the plate to be small and compact. Also, the holes for the fixing screw around the cylinder are made in a radial form centering the cylinder, realizing the hole arrangement for the plate to endure the load during bone extension. The manufacturing method for this apparatus is such that first a thick plate is drilled with a drill to make the screw holes, then a thin plate is shaved out with a milling machine leaving the height of the cylinder, to ensure the thinness and the accuracy of the holes at the same time. With the above procedures, an alveolar bone extension apparatus can be realized, an apparatus in a small and compact form securely extending one day's volume of bone extension.

### BACKGROUND OF THE INVENTION

1. Field of the Invention

This invention relates to an apparatus to conduct bone extension and to thicken alveolar bone in cases of artificial tooth root implanting, when patient's alveolar bone is too thin for an implant.

2. Description of the Prior Art

Apparatuses to extend alveolar bones do not exist currently.

### SUMMARY OF THIS INVENTION

There are many patients whose alveolar bone is thin, and therefore implanting artificial tooth root cannot be conducted due to insufficient space for implanting. Such patients had to give up conventional artificial tooth root implant installation.

This invention utilizes the natural characteristic of the bone, a bone generating characteristic of which is when two bone parts are first closely attached and then separated gradually daily from the closely attached situation, a new bone is generated between the two bone parts. This invention consists of a plate with holes for screws, an extension screw, and fixing screws, wherein the base end of the plate is fixed onto the basis of the alveolar bone with fixing screws, and a movable bone fragment is fixed onto the upper end of the plate with fixing screws. A cylinder to let through the extension screw stands like a chimney from the upper end of the plate. Thread cutting is applied at the inside of the cylinder, for the extension screw inserted into the cylinder to rotate and press the alveolar bone bases through the hole of the movable bone fragment, forcing a small separation between the alveolar bone and the movable bone fragment, the space of which will be filled according to the bone generation characteristic, thence realizing the thickening of the alveolar bone.

The tip of the extension screw can be made soft and round, or covered with soft resin cap, or in a rotating style, to prevent the tip of the extension screw from digging into the alveolar bone basis but instead to solicit the bone extension.

The pitch of the thread cutter function of the extension can be set according to the desired bone extension quantity for each day.

The holes for the fixing screws made on the plates on the alveolar bone basis are arranged so that odd number row holes and the even number row holes are lined alternately, the two rows of holes having a very short distance in between to enable the size of the plate to be small and compact. Also, the holes for the fixing screws in the circumference of the cylinder should be made in a radiating style, so that the plate is endurable for the load during the bone extension.

The manufacturing method for the plate part is, when the material is made of metal, holes are made with a drill on a thick plate, to acquire the thinness and the hole accuracy at the same time, and then the thin plate is shaved out with a milling machine leaving the height of the cylinder part.

### DESCRIPTION OF THE PREFERRED EMBODIMENTS

Hereafter, a description of the preferred embodiments of this invention is made in reference to drawings. FIG. 1 shows a pure titanium thin plate 1 the base end of which is fixed onto the alveolar bone basis with fixing screw 3a. Depending on the surface condition of the alveolar bone, the location for fixing and the number of fixing screws change accordingly. Fixing screws 3a and 3b could be made of titanium, but if absorptive material such as poly lactic acid or poly glycol acid are utilized, the extraction of the plate will be easier. Movable bone fragment 5 is fixed onto the upper end of the plate with fixing screw 3b, and the cylinder 4 to let through extension screw 2 from the upper end part of the plate 1 like a chimney. The inside of cylinder 4 has thread cutting applied, and therefore when extension screw 2 is inserted into cylinder 4 and rotated, the alveolar bone basis 6 is pressed through the hole in movable bone fragment 5, and the alveolar bone will be extended by separating the movable bone fragment 5. The tip of extension screw 2 is made soft and round, so that the tip end of the screw does not dig into the alveolar bone basis 6 even when the screw is rotated, and thus the bone extension is enabled. The tip end of the screw could be covered with soft resin cap or could be made as a rotating type. The thread cutting pitch of extension screw 2 is set so that when the screw is rotated the bone should extend for one day's quantity. The diameter of the screw head of the extension screw 2 is the same as the diameter of the thread cutting valley part diameter, so when the screw is rotated, it inserts into the inside of cylinder 4. The hole arrangement of plate 1 is made so that there is a row of three holes for the fixing screw 3a to fix the plate onto alveolar bone basis 6, and a row of four holes, arranged alternately, the row distance being short to enable the size of the plate to be small and compact. Also, the holes for the fixing screw 3b surrounding the cylinder 4 is arranged in a radial style centering the cylinder 4, and by fixing the fixing screw 3b around the cylinder, the load can be endured even when the extension screw 2 presses the alveolar bone basis 6. After the extension of the alveolar bone, the plate is removed after a certain fixing period, and at the same time, artificial tooth root implant surgery is conducted. The manufacturing method for the plate 1 part is, at first, holes are made with a drill on a thick plate and then a thin plate is shaved out with a milling machine leaving the height of the cylinder part, to acquire the thinness and the hole accuracy at the same time.

As explained above, this invention enables implanting of artificial tooth root even when the alveolar bone is thin, after extending the bone and thickening the alveolar bone. The plate is thin, made of bio-adaptable material, and by arranging the location of the holes for the screws, the apparatus can be designed to be in a small and compact size with least uncomfortable feeling on the part of the patient when the plate is implanted inside the mouth. The tip end of the extension screw is processed as soft and round, or covered with soft resin cap, or in a rotating style, so that the tip of the extension screw does not dig into the alveolar bone basis but the bone extension can be obtained securely. The thread cutting pitch of the extension screw is set to match the bone extension quantity for each day, and the extension volume can be confirmed visually. The holes for the fixing screws surrounding the cylinder are made in a radial form centering the cylinder, to enable the plate to endure the load during bone extension. The manufacturing method of the plate is that first a thick plate is drilled to make the holes, then the thin plate is shaved out with a milling machine leaving the height of the cylinder, to ensure the thinness and the accuracy of the hole arrangement at the same time.

### BRIEF DESCRIPTION OF THE DRAWINGS

FIG. 1 is a general diagram of the alveolar bone extension apparatus.

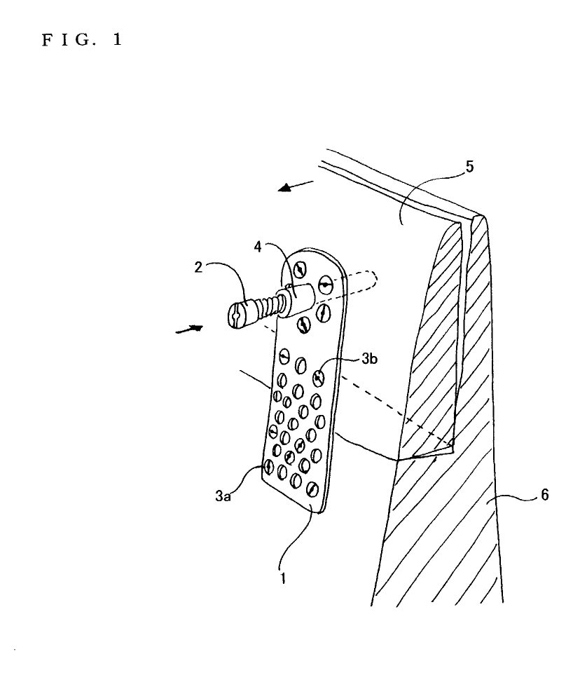

What is claimed is:

1. An alveolar bone extension apparatus that extends the bone and thickens the bone when the alveolar bone is too thin for implant in cases of implanting artificial tooth root, the apparatus consisting of a plate with holes for screws, an extension screw, and fixing screws, wherein the lower end of the plate is fixed onto the alveolar bone basis with fixing screws, a detached movable bone fragment is fixed onto the upper end of the plate with fixing screws, a cylinder to let through the extension screw extruding from the plate upper end like a chimney, the inside of the cylinder having thread cutting applied, so when the extension screw inserted into the cylinder is rotated the alveolar bone basis is pressed though the hole on the movable bone fragment, and as the alveolar bone separates from the movable bone fragment enabling bone extension.

2. An alveolar bone extension apparatus as claimed in claim 1, wherein the tip end of the extension screw is made soft and round, or covered with soft resin cap, or in a rotating style, to avoid the tip end of the extension screws from digging into the alveolar bone basis when the extension screw is rotated, ensuring the bone extension.

3. An alveolar bone extension apparatus as claimed in claim 1 and 2, wherein the thread cutting pitch of the extension screw is set to match one day's quantity of desired bone extension.

4. An alveolar bone extension apparatus as claimed in claim 1 and 2, wherein the screw holes on the plate are arranged in such a way that the odd number holes and the even number holes for the fixing screws which fix the plate onto the alveolar bone basis are arranged alternately, making the distance between the two rows of holes to be short enabling the size of the plate to be small and compact, and also the holes for the fixing screws around the cylinder to be arranged in a radial form, to let the plate endure the load during the bone extension, and the pitch of the thread cutting of the extension screws to match one day's quantity of desired bone extension volume.

5. The manufacturing method of an alveolar bone extension apparatus as claimed in claim 4, wherein if the material of the plate is metal, the method for forming such plate being, first to drill holes on a thick plate with a drill, then shave out a thin plate with a milling machine leaving the height of the cylinder, to ensure the thinness and the hole accuracy at the same time.
